(19) United States
(12) Patent Application Publication
(10) Pub. No.: US 2021/0134478 A1
(43) Pub. Date: May 6, 2021

(54) HIGH DIELECTRIC STRENGTH INSULATOR

(71) Applicant: Malcolm Robert SNOWBALL, Essex (GB)

(72) Inventor: Malcolm Robert SNOWBALL, Essex (GB)

(21) Appl. No.: 16/482,352

(22) PCT Filed: Feb. 1, 2018
(86) PCT No.: PCT/GB2018/050292

§ 371 Date: Jul. 31, 2019

(30) Foreign Application Priority Data
Feb. 1, 2017 (GB) 1701697.3
Feb. 1, 2018 (GB) 1801638.6

(51) Int. Cl. H01B 3/02; H01B 17/56; H01B 19/04; H05H 1/46; A61L 2/26; A61L 2/14; A61L 2/20

(52) U.S. Cl. CPC H01B 3/02; H01B 17/56; H01B 19/04; H05H 1/46; A61L 2202/11; A61L 2/14; A61L 2/202; A61L 2202/23; A61L 2/26

(57) ABSTRACT

A high dielectric strength insulator for use in insulating an electrode for a cold plasma generator, the high dielectric strength insulator comprising a base material having a high dielectric strength of at least 70 kV/mm, and a coating layer formed on the base material, wherein the coating layer is at least one of: formed from a material having a dielectric strength equal to or greater than the base material, formed from a material having a surface hardness greater than that of the base material, and non-porous.

### FIELD OF THE INVENTION

The invention relates to methods and apparatus to produce high dielectric strength insulators for high voltage applications particularly but not exclusively in the field of cold plasma.

### BACKGROUND OF THE INVENTION

In high voltage applications it is imperative that any insulators used can withstand the applied voltages over long periods of time and this is especially important in cold plasma applications. The voltages in cold plasma are generally very high coupled with high frequencies creating a great deal of electrical stress on the electrode holder insulator. Erosion of the insulator from the cold plasma further complicates the insulator problem.

For applications of cold plasma in air, of which there are potentially many, it is often desirable to operate at 35 kV-45 kV and at or above frequencies of 20 kHz-100 kHz, making the selection of a suitable insulator very difficult or sometimes impossible.

As a consequence of these problems the application of cold plasma as a technology has often been restricted to lower voltage applications.

Several materials have been investigated for their suitability for cold plasma applications but all have been found to be lacking because of various problems; for example:

Quartz—this material is very difficult to machine without causing micro-cracking which can lead to age stress cracking and therefore electrode insulator failure. Quartz also does not meet the desired ac dielectric strength.

Mica—this material is difficult to machine into complex shapes and is not mechanically robust enough for most applications. It also does not have a sufficiently high ac dielectric strength for the purposes of cold plasma generation.

### SUMMARY OF THE INVENTION

Desired criteria for a reliable cold plasma insulator include:
1) Very high ac dielectric strength, preferably above 70 kV/mm.
2) Low dielectric constant and low loss factor to prevent high frequency dielectric heating.
3) Able to be machined into complex forms.
4) Have high tracking resistance under high voltage conditions.
5) Must be non-porous and water resistant for high humidity applications.
6) Must resist erosion from cold plasma.
7) Be hard wearing for electrode heads performing repetitive applications.

Boron Nitride meets most of the criteria especially when it is hot pressed, parallel to the processing direction. Unfortunately it suffers from two major problems which make it unsuitable. These problems are that:
(1) the material is soft, porous and absorbs water reducing its ac dielectric strength to an unacceptable level over time.
(2) the material is easily mechanically abraded. Boron nitride is one of those materials which has superb features yet also has a major failing which has severely curtailed its use in engineering and science.

It will be understood that the dielectric strength is the maximum electric field that a material can withstand under ideal conditions without breaking down (i.e., without experiencing failure of its insulating properties). It will also be understood that a low dielectric constant may have a small dielectric constant relative to silicon dioxide, SiO₂, which has a dielectric constant of 3.9, whereas a high dielectric constant may have a large dielectric constant relative to silicon dioxide. Dielectric loss (loss factor) quantifies a dielectric material's inherent dissipation of electromagnetic energy (such as heat). It can be parameterized in terms of either the loss angle δ or the corresponding loss tangent tan δ. Both refer to the phasor in the complex plane whose real and imaginary parts are the resistive (lossy) component of an electromagnetic field and its reactive (lossless) counterpart.

After intensive research the inventor has concluded that there is not one single material which meets the desired criteria for a reliable cold plasma insulator and therefore a composite solution is desired which involves a combination of materials and a specific production process.

The invention consists of a combination of materials and the process to produce the materials in a synergistic way to meet the demanding criteria desired.

A base material, such as boron nitride, is machined into a final shaped base material, and then the final shaped base material is coated with another coating material, to form a coating layer as a thin film layer (for example, with a thickness ranging from fractions of nanometres, to several micrometres). This may solve the shortcomings of the boron nitride whilst still allowing it to maintain its advantageous specification, such as its high dielectric strength and machinability. The combination of the materials may enable the finished product to fully meet the desired criteria.

Under high voltage conditions the coating layer should meet the following coating process criteria:
1) The coating layer should at least have the same ac dielectric strength as the boron nitride base material so that it does not lower the surface resistivity of the boron nitride leading to tracking under high voltages at the base to coating interface.
2) The coating process must apply the coating such that it is stress free and will not age crack.
3) The coating process must lay the coating down such that it is smooth, of consistent thickness and does not contain any inclusions which will cause tracking paths for the high voltage.

It will be understood that the coating layer may be applied as a monolayer or as a multilayer.

Boron Nitride for example hexagonal boron nitride, for example with boric oxide binder pressed in the parallel orientation (grade BO), available for example from Accuratus Corporation (35 Howard Street, Phillipsburg, N.J., 08865, USA), may have mechanical, thermal and electrical properties with the following approximate values (which may be found for example at http://accuratus.com/pdf/BNBOprops.pdf):

Orientation Relative
to Pressing Direction

| | |
|---|---|
| Parallel | Perpendicular |

Thermal

| | | |
|---|---|---|
| Thermal conductivity (W/m · K) | 30 | 33 |
| Coefficient of Thermal Expansion | 11.9 | 3.1 |
| (10⁻⁶/° C.) | | |
| Specific heat (J/Kg · K) | 1610 | |

Electrical

| | | |
|---|---|---|
| Dielectric Strength (ac-kv/mm) | 95 | 79 |
| Dielectric Constant (at 8.8 GHz) | 4.6 | 4.2 |
| Dissipation Factor (at 8.8 GHz) | 0.0017 | 0.0005 |
| Volume Resistivity (ohm · cm) | >10¹⁴ | >10¹⁵ |

Mechanical

| | | |
|---|---|---|
| Density (gm/cc) | 1.9 | 1.9 |
| Porosity (%) | 2.8 | 2.8 |
| Flexural Strength (MPa) | 75.8 | 113 |
| Elastic Modulus (GPA) | 46.9 | 73.8 |
| Compressive strength (MPa) | 143 | 186 |
| Hardness (Kg/mm²) | 15-24 | 15-24 |
| Maximum Use Temperature (° C.) | 1800 | |

This or equivalent/similar forms of boron nitride may meet most of the criteria desired for the base material namely:
1) Very high ac dielectric strength (95 kV/mm).
2) Low dielectric constant and dissipation factor.
3) Able to be machined into complex forms.
4) Have high tracking resistance under high voltage.
5) Does not need special cutting tools

Boron nitride (grade BO) also has a dielectric constant (at 8.8 GHz) of 4.6 in an orientation parallel to the pressing direction and a dissipation factor (at 8.8 GHz) of 0.0017 in an orientation parallel to the pressing direction. It will also be understood that these values and the value of the dielectric strength are at room temperature and pressure.

Unfortunately as explained previously the boron nitride is porous (e.g. 2.8% in an orientation parallel to the processing direction for “BO grade” boron nitride) and absorbs water and as a consequence its dielectric strength drops to an unacceptable level. It is however a soft material (hardness of 15-24 kg/mm² or 0.15 GPa at room temperature in an orientation parallel to the processing direction) and is therefore very susceptible to abrasion wear.

Manufacturers have attempted to solve the porosity problem by adding silica to the boron nitride and then hot pressing the material. However, the porosity problem is solved at the expense of ac dielectric strength which drops to approximately 58 kV/mm, making this material unsuitable for use as a reliable cold plasma insulator.

### Cubic Boron Nitride

This material meets most of the desired criteria for the base material but as it is the second hardest material known in science (with a Knoop hardness of 45 GPa compared to a Knoop hardness of 100 GPa for diamond, and with a Mohs hardness of around 9.5 to 10) it is both time-consuming to work this material, difficult to machine it into complex forms and it requires special machining tools.

Preferably Boron Nitride with boric oxide binder pressed in the parallel orientation is the base material.

Research and development carried out by the inventor has identified the following potential materials to coat the parallel pressed boron nitride.

Very High Purity SiO₂ (99.99999% pure)

This material meets the coating criteria with a very high ac dielectric strength of 100 kV/mm and is able to be coated onto materials using a number of coating processes. This material can be used to coat the boron nitride and this material combination meets the desired criteria for use as a reliable cold plasma insulator mentioned above. SiO₂ in its ceramic form has a Knoop hardness of 6.92 GPa if parallel to the optical plane and 7.75 GPa if normal to the optical plane, and has a Mohs hardness of 7 (compared to a Mohs hardness of 10 for Diamond), indicating its ability to resist scratches.

In practice this purity of SiO₂ is difficult to obtain and is therefore expensive. SiO₂ has to be fired at 1600° C.-1900° C. in an inert atmosphere to complete the coating process, then allowed to cool in the furnace to remove stresses.

It is very difficult to keep this level of purity throughout the coating process as the smallest amount of contamination significantly reduces the dielectric strength making it unsuitable.

### Diamond and Synthetic Diamond, Including Polycrystalline Diamond.

This material meets all of the desired criteria for a coating and when used with boron nitride as a base material meets the desired criteria for use as a reliable cold plasma insulator mentioned above. It gives an extremely high ac dielectric strength of 330 kV/mm. It is waterproof (>2 μm coating) and very resistant to high voltage tracking; diamond can be economically applied using a number of coating processes such as chemical vapour deposition (CVD) and more especially using the latest laser deposition processes developed by QQC Inc., for example as described in U.S. Pat. Nos. 6,203,865, 5,620,754 (A) and 5,731,046 (A). Diamond is also a low loss material and meets all of the abrasion criteria as well as being resistant to cold plasma erosion.

Preferably diamond or synthetic diamond is used as a coating layer to coat parallel pressed boron nitride to provide a reliable cold plasma insulator.

Because of the very high ac dielectric strength (95 kV/mm) of the combination of parallel pressed boron nitride coated with a diamond coating, this allows insulators and therefore electrodes to be made much smaller. Applications of the invention significantly open new applications for boron nitride.

Aspects of the invention are as set out in the independent claims and optional features are set out in the dependent claims.

In an aspect there is provided a high dielectric strength insulator for use in insulating an electrode for a cold plasma generator, the high dielectric strength insulator comprising a base material having a high dielectric strength of at least 70 kV/mm, and a coating layer formed on the base material, wherein the coating layer is at least one of: formed from a material having a dielectric strength equal to or greater than the base material, formed from a material having a surface hardness greater than that of the base material, and non-porous.

In an aspect there is provided a method of manufacturing a high dielectric strength insulator, the method comprising forming a base material from boron nitride; coating the base material with a coating layer while the base material is held at an elevated temperature to inhibit moisture absorption, wherein the coating layer is at least one of: formed from a material having a dielectric strength equal to or greater than the base material, formed from a material having a surface hardness greater than that of the base material, and non-porous.

In an aspect there is provided a system for cold plasma generation, the system comprising an electrode, and an insulator comprising a base material having a high dielectric strength of at least 70 kV/mm and a coating layer formed on the base material, wherein the coating layer is at least one of: formed from a material having a dielectric strength equal to or greater than the base material, formed from a material having a surface hardness greater than that of the base material, and non-porous.

Aspects of the invention may be provided in conjunction with each other and features of one aspect may be applied to other aspects.

### BRIEF DESCRIPTION OF THE DRAWINGS

FIG. 1—Shows a schematic diagram of a process for producing a coated boron nitride material suitable for use a high voltage dielectric insulator for use with cold plasma generation.

FIG. 2—Shows a section through a typical high voltage insulator made from the coated boron nitride material.

FIG. 3—Shows a section through a typical cold plasma electrode holder made from the coated boron nitride material.

FIGS. 4A-F—Show an example cold plasma generation electrode holder comprising an insulator.

FIG. 5—Shows a schematic view of an example insulator with a shield layer.

### SPECIFIC DESCRIPTION

Embodiments of the present invention will now be described by way of example only with reference to the accompanying drawings.

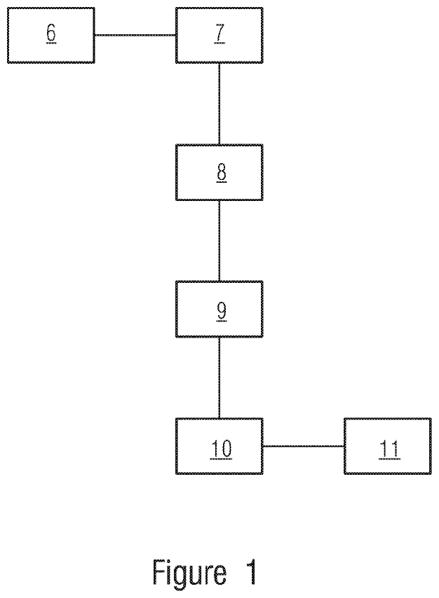

### FIG. 1

Referring to FIG. 1, a process for producing a high voltage dielectric insulator suitable for use with cold plasma generation is described.

Machining Operation 7

Raw boron nitride material 6, e.g. hexagonal boron nitride, such as grade BO boron nitride, is machined into its finished base shape 3 using normal tool steel cutting tools.

Preferably all sharp corners are removed to inhibit the formation of high electromagnetic fields which cause high stress points in the material under high voltage conditions.

Preferably linear tracking paths are made long by including ripples and undulations in the surface of the insulator.

Cleaning Operation 8

The boron nitride finished base 3 is thoroughly washed and degreased to ensure that all contamination is removed from the surface of the boron nitride.

Preferably the cleaning fluids are non-aqueous in composition to minimise water absorption by the boron nitride.

Preferably the cleaning fluid is chemical based which completely volatises and leaves no residue after drying.

Preferably the finished final shape is thoroughly cleaned in an ultrasonic bath.

Baking Operation 9

All traces of water must be removed from the boron nitride and therefor the finished article is baked for a period of time at an elevated temperature.

Preferably the boron nitride finished base 3 is baked at 130° C. for 30 minutes.

Preferably the boron nitride finished base 3 is baked at 150° C. for 30 minutes.

The finished article is maintained an elevated temperature until it is moved into the next stage to make sure that the finished article does not reabsorb any water.

Preferably the holding temperature is a minimum of 110° C.

Coating Operation 10

The finished article is coated with the selected coating using a proprietary commercial coating process, such as a Laser Deposition Process developed by QQC Inc, which meets the coating process desired criteria outlined above.

Preferably the coating process is Chemical Vapour Deposition (CVD). In CVD the object to be coated is placed in a chamber containing a high-pressure, high-temperature mixture of methane, or some other carbon-based gas, and hydrogen. The gases are heated by hot filaments or radio waves, breaking up the methane into its constituent carbon and hydrogen atoms. The electrically charged carbon atoms settle on the object to be coated, the majority of them arranging themselves as crystalline diamond, rather than as graphite, another crystalline form of carbon. The coating is of a consistent thickness and is free from inclusions.

Preferably the coating process is a Laser Deposition Process developed by QQC Inc. (as described above) which can deposit a diamond coating very quickly at room temperature if desired and in air. The process uses a number of lasers which when multiplexed break up the carbon dioxide in the air into constituent parts of carbon and oxygen. The substrate to be coated is placed in the vapour created by the lasers and the carbon atoms in the vapour are deposited onto the surface of the substrate using a laser which scans the surface of the substrate. The consequential deposited surface coating is pure diamond. The coating is of a consistent thickness and is free from inclusions. The deposition rate for this process is 1 micron per second, which is approximately 1,000-3,000 times faster than the CVD process. To preserve the boron nitride's water-proofing it is vital that the whole of the surface of the material is coated with the diamond coating including any holes, grooves etc. This process forms a chemical bond to the boron nitride at the atomic level giving a very high adhesion to the coating.

Work done by the inventor shows that the film thickness should preferably be a minimum of 2 μm. Preferably the film thickness should be 10 to 30 μm to achieve a hard abrasion-resistant surface.

Testing Operation 11

The finished insulator is tested for compliance with the desired criteria as described above.

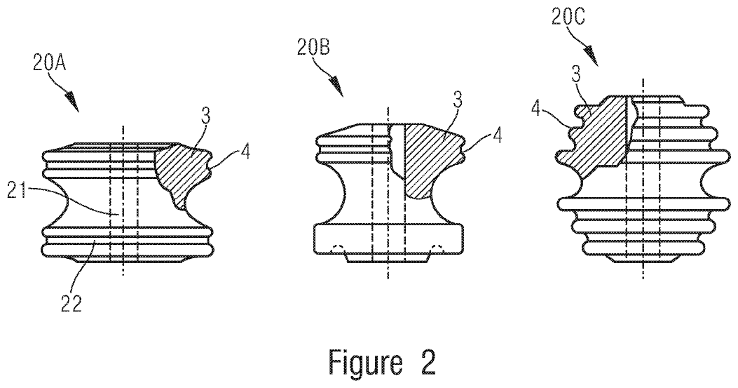

FIG. 2 shows a partial section view through a selection of coated insulators 20A-C, which may be manufactured by the process described above with respect to FIG. 1. It will be appreciated that the examples shown in FIG. 2 are merely exemplary, and that other insulator shapes are envisaged. In the examples shown in FIG. 2, the insulators 20A-C comprise a base material 3, such as boron nitride, for example BO grade boron nitride or equivalents thereof.

The base material 3 of high dielectric strength insulators 20A-C may have been machined according to precision ceramic techniques, such as the processes described in relation to FIG. 1. In this way symmetrical and substantially cylindrical insulators may be formed with a plurality of grooves such as ripples or undulations on their surface. The surface grooves may inhibit tracking, for example by lengthening the path of any linear tracking lines on the surface of the insulator.

The insulators 20A-C comprise a coating layer 4 that coats the base material 3, for example diamond or synthetic diamond. The coating layer 4 forms a thin film layer to coat the base material 3, for example according to the processes described herein in relation to FIG. 1. In preferred examples, the film thickness of the coating layer 4 is a minimum of 2 μm, and preferably the film thickness is 10 to 30 μm. Such a film thickness of the coating layer 4 may ensure that the surface of the coating layer is hard and/or abrasion-resistant.

The coating layer 4 has an ac dielectric strength that is at least as high as that of the base material 3, for example which may ensure that it does not lower the surface resistivity of the boron nitride. The coating layer 4 may be non-porous and/or waterproof, for example to inhibit absorption of water and/or oxygen in the air by the base material 3, which may reduce the ac dielectric strength of the base material e.g. in high humidity applications. The coating layer has a high surface hardness, for example greater than GPa, for example greater than or equal to 100 GPa. This may inhibit abrasion and/or erosion of the base layer, which may otherwise occur during cold plasma generation.

Each of the insulators 20A-C comprise a cavity 21 along their central axis into which an electrode may be inserted, such as an electrode for a cold plasma generator. Each of the insulators 20A-C may thereby form an insulating electrode head for an electrode, to electrically insulate the electrode during its operation.

As can be seen in the FIG. 2, the insulators 20A-C have smooth surfaces without sharp edges or corners. This may inhibit the formation of high electromagnetic fields which cause high stress points in the material under high voltage conditions during operation of the electrode.

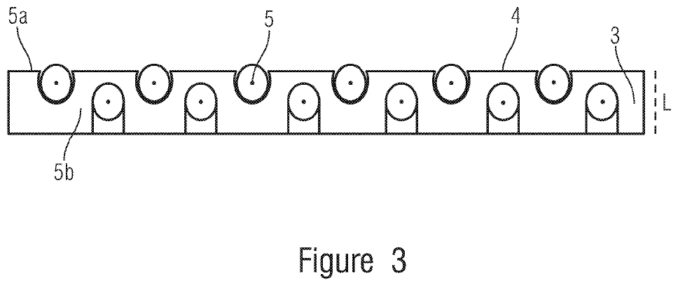

FIG. 3 shows a partial section side view through an example cold plasma insulator 300 comprising a base material 3 and a coating layer 4. For example the insulator 300 may be a diamond coated boron nitride cold plasma electrode insulator 300 manufactured by the processes described herein, for example the processes described in relation to FIG. 1. In the example shown in FIG. 3, the boron nitride base material 3 is coated with a diamond coating layer 4, and a plurality of electrodes 5 are embedded into the insulator. In particular two interdigitated sets of electrodes 5a and 5b are positioned within the insulator 300. The first set of electrodes 5a are positioned in a series of parallel channels in the top surface of the insulator 300, such that a portion of the insulators 5a are open to the air. The second set of insulators 5b are positioned centrally within the insulator 300, within a series of parallel cavities in the bottom surface of the insulator 300. The electrodes in each set are connected to a common power line. The electrodes in each set are substantially cylindrical, and extend from their respective power line parallel to and in the same plane as the other electrodes in their set. The electrodes in the first set may extend from their common power line towards the common power line of the second set, and vice versa. The first and second set of electrodes may be offset from one another in a plane of the insulator 300, for example the first and second set may be offset along the axis L that runs between and perpendicular to the top surface and bottom surface of the insulator 300. In other examples the first and second set of electrodes aligned in the axis L of the insulator, such that the first and second sets of electrodes are interdigitated with one another in the same plane.

Although two sets of electrodes are shown in FIG. 3, it will be appreciated that in other examples the insulator could accommodate a greater or fewer number of sets of electrodes.

The electrodes 5a, 5b of FIG. 3 are each connected to a high voltage AC power supply via connections (not shown). In operation, a voltage is applied to the electrodes 5. In response to this voltage exceeding a threshold voltage, cold plasma generation may occur between the electrodes. For example plasma may be generated between a first electrode in the first electrode set 5a and its neighbouring electrodes in the second electrode set 5b.

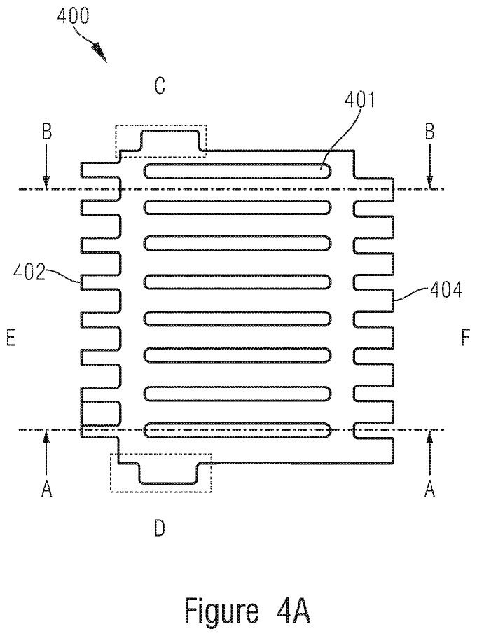

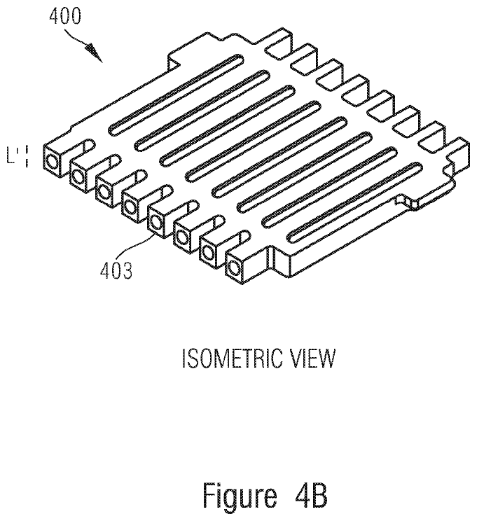

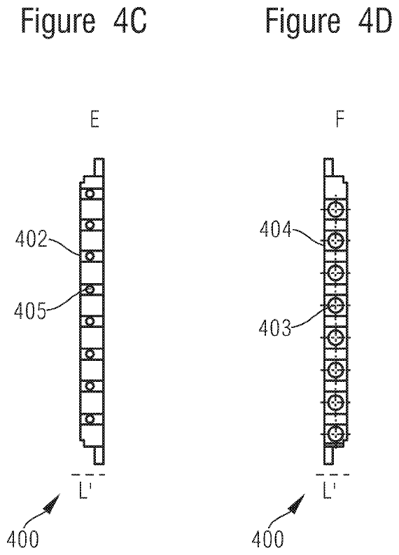

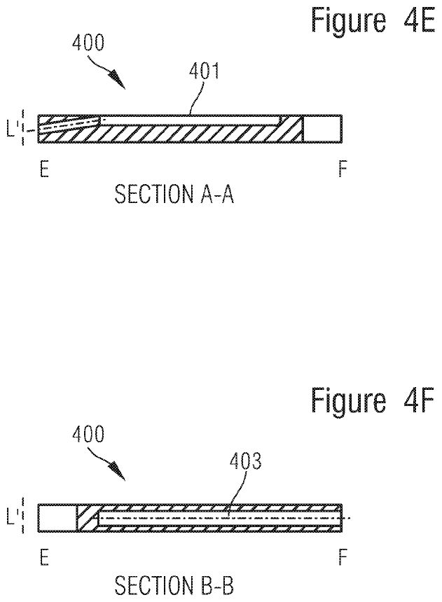

FIG. 4A shows a plan view of the top surface of an example of an electrode holder 400 that comprises an insulator such as the insulator described herein. For example an insulator such as that shown in FIG. 3 may form part of the electrode holder shown in FIGS. 4A-F. A plurality of electrodes (not shown) may be inserted into the holder 400, and the holder may provide electrical insulation for the electrodes during their operation, such as during cold plasma generation. A corresponding isometric view of the electrode holder 400 is shown in FIG. 4B. The top surface of the holder 400 is substantially rectangular, comprising teeth, channels, and cavities as described below. Three axes of the holder can be defined: a first axis that extends from the top surface of the holder to a bottom surface; a second axis that extends from a first end C to a second end D and a third axis that extends from a first side E to a second side F.

The top surface of electrode holder 400 comprises a series of parallel channels 401, which are equally spaced along the second (CD) axis of the holder 400. The channels extend parallel to one another along the third (EF) axis such that along a central portion of the EF axis, a top section of the first electrodes are exposed. The channels are arranged so that a first set of electrodes can be positioned in them. This first set of electrodes are connected to a high voltage AC power supply via connectors positioned in a first set of teeth 402 of the holder, that are aligned with the channels 401. For example each connector may connect a first electrode to a common first power line.

Electrode holder 400 further comprises a series of parallel cavities 403, which are equally spaced along the second (CD) axis of the holder 400. The cavities 401 extend parallel to one another along the third (EF) axis of the holder 400. The cavities 401 are positioned centrally between the top and bottom surfaces of the holder 400, for example centrally along the length L′ shown in FIG. 4B. The cavities 403 are arranged so that a second set of electrodes can be positioned in them. This second set of electrodes are connected to a high voltage AC power supply via connectors positioned in a second set of teeth 404 of the holder, that are aligned with the cavities. For example each connector may connect a second electrode to a common second power line.

The second set of electrodes may be positioned in a series of equally spaced parallel cavities 403 within the holder 400, for example along the line B-B, such that they are interleaved or interdigitated with the first set of electrodes. The second set of electrodes are connected to the AC power supply via connectors positioned in a second set of teeth that are aligned with the cavities 403. The electrodes in each set are connected to a common power line via their connectors. The electrodes in each set are substantially cylindrical, and extend from their respective power line parallel to and in the same plane as the other electrodes in their set. For example the electrodes in the first set may extend from their common power line towards the common power line of the second set, and vice versa. The first and second set of electrodes may be offset from one another in a plane of the holder, for example the first and second set may be offset along the axis L′ that runs perpendicular to the top surface and bottom surface of the holder 400.

In other examples the first and second set of electrodes may be parallel to one another and aligned in the axis L′ of the holder, e.g. so that they interdigitated in the same plane. For example the second set of electrodes may be positioned in channels in the top surface of the holder, or the first set of electrodes may be arranged in cavities positioned centrally between the top and bottom surfaces of the holder.

FIG. 4C shows a side view (E) of the holder 400, showing a first set of teeth 402, each comprising an opening 405 which leads to the channels 401 into which the first set of electrodes can be positioned. The first set of teeth 402 are arranged to enable first electrical connectors to couple the first set of electrodes to a first power line. The first set of teeth may be arranged to insulate the first electrical connectors and/or to prevent the connectors from being damaged by an external force.

FIG. 4D shows another side view (F) of the holder 400, showing a second set of teeth each comprising an opening to one of the cavities 403, into which the second set of electrodes can be positioned. The second set of teeth 404 are arranged to enable second electrical connectors to couple the second set of electrodes to a second power line. The second set of teeth may be arranged to insulate the second electrical connectors and/or to prevent the connectors from being damaged by an external force.

FIG. 4E shows a sectional view of holder 400 along the line A-A shown in FIG. 4A, showing the channels 401 into which the first set of electrodes can be positioned. The channel 401 is open to the air at the top surface of the holder 400 and is surrounded on all other sides by an insulator, for example the high dielectric strength insulator described herein. Such an arrangement of the electrodes may help to direct a generated field and/or plasma in a particular direction.

FIG. 4F shows a sectional view of holder 400 along the line B-B shown in FIG. 4A, showing the cavities 403 into which the second set of electrodes can be positioned. The cavity 403 has an opening on the side F of the holder and is surrounded on all other sides by an insulator, for example the high dielectric strength insulator described herein.

The first and second set of teeth 402, 404, protrude from the sides E, F of the holder 400 respectively. In some systems, multiple holders may be coupled together, for example by interdigitating the first set of teeth 402 of one holder with the second set of teeth 404 of another holder, to form a larger insulator/electrode system.

Although the examples shown in FIGS. 2 and 3/4 show two possible applications, there are many; in fact an insulator for any application for which a reliable high voltage is desirable can be produced using the processes described herein.

Another desirable feature of the combination of boron nitride coated with diamond is that a final insulator can work at a very high temperature, such as in excess of 750° C., for applications where the insulator is desired to work in high voltage; high temperature conditions (arc furnaces, the nuclear industry, plasma physics etc.). This ability to work at high temperatures further expands the applications of dielectric insulators manufactured from a boron nitride base material and coated with diamond.

Boron nitride (BO grade) has working temperature of 1200° C. in an inert atmosphere and 850° C. in air. Diamond, as well as synthetic diamond, has a working temperature of 950° C. in air, therefor an insulator made according to the invention process will operate at 950° C. in air.

It is noted that high concentrations of oxygen are found in some ozone generators. The coating layer (for example, diamond and/or synthetic diamond) may be susceptible to attack and erosion from such high concentrations of oxygen. To address these problems, in some examples the insulator may be coated in a shield layer, such as a layer of SiO₂, such as pure SiO₂ (for example, 99.99999% pure), which may act to shield the coating layer from the oxygen while maintaining the dielectric strength of the coating layer and/or the base material. The shield layer may be applied over the coating layer, for example using the Sol-gel process and/or CVD processes. The shield layer may be applied as a thin film layer, for example, with a thickness ranging from fractions of nanometres, to several micrometres. In some examples the shield layer is at least 2 μm thick, and in some examples may be between 10 to 30 μm thick. The shield layer may comprise a monolayer of material or may comprise a plurality of layers of material. The inventors have discovered that if the SiO₂ is coated onto the diamond or synthetic diamond, for example using the Sol-gel process and/or CVD processes, the SiO₂ can be of a sufficient purity so that the dielectric strength of the insulator is not compromised. The shield layer material may be a different material to the coating layer material.

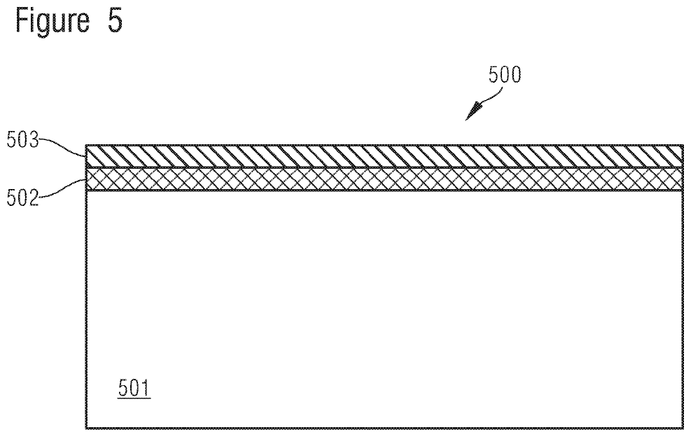

FIG. 5 shows a schematic view of an insulator 500 comprising a base material 501, a coating layer 502, and a shield layer 503. The base material 501 may comprise boron nitride, for example BO grade boron nitride. The coating layer 502 may comprise diamond or synthetic diamond. The coating layer 502 may form a thin film layer on the surface of the base material 3, for example by the process described above in relation to FIG. 1. The shield layer 503 may comprise layer of SiO₂, such as pure SiO₂ (for example, 99.99999% pure), which may be applied over the coating layer 4 according to the processes described.

The coating layer 502 forms a thin film layer to coat the base material 501, for example according to the processes described herein. In some examples the coating layer 502 is diamond or synthetic diamond. The coating layer 502 may be applied as a thin film layer, for example, with a thickness ranging from fractions of nanometres, to several micrometres. In some examples the shield layer is at least 2 μm thick, and in some examples may be between 10 to 30 μm thick. The coating layer 502 may comprise a monolayer of material or may comprise a plurality of layers of material. Such a film thickness of the coating layer 4 may ensure that the surface of the coating layer is hard and/or abrasion-resistant. The coating layer 501 is also non-porous and/or waterproof, for example to inhibit absorption of water and/or oxygen in the air by the base material 3, which may reduce the ac dielectric strength of the base material e.g. in high humidity applications.

The insulator 500 may be further coated in a shield layer 503. The shield layer 503 may be a layer of SiO₂, such as pure SiO₂ (for example, 99.99999% pure). The shield layer is applied over the coating layer 502. This may be achieved using the Sol-gel process and/or CVD processes. The shield layer 503 may be applied as a thin film layer, for example, with a thickness ranging from fractions of nanometres, to several micrometres. In some examples the shield layer 503 is at least 2 μm thick, and in some examples it may be between 10 to 30 μm thick. The shield layer 503 may comprise a monolayer of material or may comprise a plurality of layers of material.

The shield layer 503 may be arranged to shield the coating layer from oxygen while maintaining the dielectric strength of the coating layer 502 and/or the base material 501. For example the shield layer 503 may protect that coating layer 502 and/or the base material 501 by inhibiting attack and erosion by oxygen that the insulator may otherwise be susceptible to in environments that contain high concentrations of oxygen, such as during ozone generation.

The shield layer 503 may be non-porous and/or impermeable to water, e.g. waterproof, to inhibit the penetration of water to the coating layer 502 and/or the base material 501. The shield layer may in this way inhibit adverse effects caused by water, such as the erosion of the insulator 500 or inhibit any loss of its dielectric strength, which may otherwise b for example in high humidity applications.

Although FIG. 5 shows the thicknesses of the coating layer and shield layer to be the same as one other, it will be appreciated that this is merely exemplary and the coating layer and shield layer may differ from one another and may each have a range of different thicknesses, for example a thickness in the range of thicknesses described above.

Although not shown in the Figures, such a shield layer may be applied, by the processes described, to the example insulators shown in FIGS. 2-4.

With reference to the drawings in general, it will be appreciated that schematic functional block diagrams are used to indicate functionality of systems and apparatus described herein. It will be appreciated however that the functionality need not be divided in this way, and should not be taken to imply any particular structure of hardware other than that described and claimed below. The function of one or more of the elements shown in the drawings may be further subdivided, and/or distributed throughout apparatus of the disclosure. In some embodiments the function of one or more elements shown in the drawings may be integrated into a single functional unit.

The electrodes and insulators described herein may form part of an apparatus for sterilising a packaged product. For example, the apparatus may comprise a pair of electrodes, such as gas filled electrodes, means for generating a high voltage between the electrodes sufficient to create a high electromagnetic field and create a cold plasma therebetween. The apparatus may be arranged to irradiate a package containing said product with said field.

The electromagnetic field may create a cold plasma which is energetic enough to convert oxygen in air into ozone and other reactive oxygen based species. Means may be provided for directing the generated electromagnetic field towards the product to be sterilised.

Furthermore, the use of plasma may create oxidising species which have a higher oxidising potential than ozone and therefore are more efficient at killing microorganisms.

An apparatus for generating ozone inside packaged articles typically comprises an electrode assembly in which coplanar electrodes are supported along a contact surface. The electrodes may be solid state conductive electrodes and/or gas filled electrodes. These electrodes may be interdigitated and/or arranged with uniform spacing therebetween along a portion of their length. Where the electrodes are straight they may be parallel, but other shapes can also be evenly spaced. In some examples the electrodes are partially insulated and partially exposed.

Each electrode may be elongate, for example each electrode may be curved, coiled, bent or otherwise non-linear along its length. Each electrode may comprise a plurality of interconnected linear sections. Each electrode may be generally planar, said field directing means being arranged to direct the electromagnetic field perpendicular to said plane towards the product to be sterilised. The electrodes may extend side-by-side along their length and may be separated by a substantially uniform gap. The field directing means may extend on one side of the electrodes and may comprise a ferromagnetic material. The field directing means may at least partially extend between the electrodes. The field directing means may comprise a surface which is profiled to receive said electrodes.

The electrodes may be contained within an open-fronted cavity. The cavity may be defined by said field directing means. The electrodes may extend in a plane parallel to the front of the cavity. The cavity may comprise a side wall or walls which extend around the electrodes and which may be arranged to seal against the packaging of the product to be sterilised. Means may be provided for evacuating air or other gas from said cavity when the latter is sealed against the packaging of the product to be sterilised.

The high voltage generation means may produce voltages pulses in the range of 1 kV to kV. The high voltage generation means may have a constant voltage component which is of a magnitude sufficient to keep the gas within the electrodes ionised. The high voltage generation means may produce pulses of high voltage in the range 5 ns to ms duration. The high voltage generation means may be arranged to produce pulses of variable magnitude, variable width and/or variable repetition rate.

In some examples the apparatus comprises a sensor for monitoring the electromagnetic field, the sensor being connected to means arranged to vary the output parameters of said high voltage generation means. In this way, the high voltage generation means can accept a feedback signal from the electromagnetic field sensor and can automatically adjust the magnitude of the high voltage pulses and the other pulse parameters, in order to adjust the electromagnetic field and maintain it at a constant level. This ensures constant ozone production package to package. In some examples said high voltage generation means is arranged to produce voltage pulses of opposite polarities and to apply said pulses to respective electrodes. In some examples the apparatus comprises means for agitating or otherwise moving the product to be sterilised: the products may be irradiated with said electromagnetic field before, after and/or during said agitation. In some examples the agitation means is arranged to at least partially rotate the package. This approach encourages the disinfection gas to quickly permeate through the package and get to all surfaces. In some examples the apparatus is arranged to irradiate successive products. In some examples the apparatus is arranged to successively irradiate the same product.

The insulator and electrodes described herein may form part of a package disinfection. In some examples the electrodes are substantially covered with an insulating material, such as the insulator described above in relation to FIGS. 1 to 5. In some examples one electrode is covered with an insulating material and the other comprises an exposed electrically conductive region. In an example the electrodes comprise distributed impedances, and the electrodes may comprise a plurality of raised regions distributed along their length. For example the electrodes may comprise a coiled conductor and the raised regions are provided by the turns of the coil. The raised regions may comprise ridges. Adjacent raised regions may be coupled by a series impedance. Typically the transverse cross section of the electrodes is square however they may also be round, or rectangular in cross section.

The electrodes may be arranged such that, in use each electrode comprises a feed end for receiving electric current and a second end and the electrodes are arranged generally alongside each other and in opposition such that the feed end of each of the two electrodes is arranged in apposition to the second end of the other of the two electrodes. In some examples the apparatus comprises an electrode support for supporting the electrodes to enable them to be brought into contact with a package. In some examples the apparatus comprises a means for urging the electrode into contact with a package to be disinfected. The means for urging and/or the support may comprise a suction coupling to couple a suction source to a contact surface of said electrode support. The support may be an insulator as described above for example in relation to FIGS. 1 to 5.

The apparatus may also comprise a sensor for sensing pressure at the contact face of the electrode support to enable control of the current based on the pressure. The electrode support may comprise a seal or sealing member arranged on or around said contact surface. In some examples the electrodes are arranged in a substantially coplanar configuration and they may be substantially parallel. One or more electrode may be arranged in an insulating sheath, such as the insulators described above in relation to FIGS. 1 to 5. The electrodes may be embedded/potted in an insulating material to exclude air gaps from around the electrodes. The insulating material may comprise a cured material which is introduced into the sheath in liquid form.

Typically the apparatus is configured to convert oxygen to ozone by generating a plasma. The apparatus may be configured such that capacitive coupling between the electrodes promotes the conversion of oxygen to ozone within said package by means of the electric field between said electrodes. The package disinfection apparatus may comprise a low voltage AC power supply and a first step up transformer coupled to a first one of the two electrodes and a second step up transformer coupled to the other of said electrodes so that said transformers provide a power supply to said electrodes of relatively higher voltage than said low voltage AC power supply. In some examples each transformer is arranged in close proximity to the electrode to which it supplies power. The transformers may be coupled to the electrodes by shielded cables.

The package disinfection apparatus may comprise a current sensor for sensing current flow between said electrodes in order to detect an over current condition and control means for preventing operation of the packet disinfection apparatus in the event an over current condition is detected.

In some examples the apparatus is adapted for processing a plurality of packaged articles and comprises means for adjusting the voltage applied to said electrodes and/or the length of time for which said voltage is applied based on the type of article. The electrodes may be arranged less than 5 mm apart, for example less than 3 mm apart, for example substantially 2 mm apart, in some cases less.

Also described herein is a packet disinfection electrode assembly for generating plasma inside a package comprising a packaged article and an air space, the electrode assembly comprising: a dielectric head, having a contact surface for contacting said package, and which may comprise the insulator described above in relation to FIGS. 1 to 5; and at least two electrically conductive electrodes distributed about the contact surface, wherein a first one of the two electrodes is insulated, e.g. by the insulator described above, and an electrically conductive region of the second of said electrodes is exposed near the contact surface. This use of both exposed and insulated electrodes has been found to enable packages to be disinfected using substantially lower power. In some possibilities the exposed electrode may be earthed.

In some examples the spacing between adjacent edges of the first and second electrode is even along at least a portion of the length of the edges. This has the advantage of enabling reproducible and stable production of plasma in well defined regions adjacent the contact surface. In some possibilities the spacing between adjacent edges along the portion comprises the distance of closest approach of the edges and this/these portion(s) may be continuous in extent or may be broken or discontinuous and/or spread in a number of portions along the electrodes. In some possibilities the spacing between adjacent edges of the first and second electrode is less than 20 mm, for example less than 15 mm, for example less than 10 mm. In some possibilities the spacing is less than 5 mm, and may be between 1 mm and 4 mm. In some cases the electrodes are elongate and have a major dimension and a minor dimension. In some examples the electrodes are aligned along their major dimension and are less than 15 mm wide along their minor dimension. In some examples they are less than 10 mm, for example less than 5 mm wide. This has the advantage of enabling more plasma creating regions to be provided in a package of fixed size than would be possible where broader electrodes are used.

In some possibilities the first electrode is provided by a first plurality of electrodes and the second electrode is provided by a second plurality electrodes. The first plurality of electrodes may be interleaved with the second plurality of electrodes so that alternate electrodes are insulated, for example by the insulator described above in relation to FIGS. 1 to 5, whilst the respective other alternate electrodes comprise exposed conductive regions. This has the advantage of reducing the size of the electrode assembly and still further reducing the power required to establish a plasma inside a packaged article.

The electrode assembly may be used in an apparatus comprising a mechanical bias adapted to urge the contact surface against said package with a selected force. In some cases the apparatus comprises a sensor configured to sense the back pressure generated by urging the package against the contact surface and a controller configured to control the mechanical bias based on the sensed back pressure. This has the advantage that the package can be urged into close contact with the assembly without risking damage to the package. In some examples the selected force is determined by a setting of the controller, and for example this setting may be programmable.

The electrodes may be elongate and may comprise a reactive and/or resistive impedance. In some possibilities the electrodes may be arranged so that their impedance is spatially distributed across the area of the contact surface. For example, the electrodes may comprise coils.

In some possibilities the coils are embedded in the head and conductive regions of the second electrode are exposed at or near the contact surface. In some possibilities the second electrode is recessed from the contact surface and in some possibilities the second electrode is flush with the contact surface. The coils may comprise a round cross section but may comprise at least one straight side or be square or rectangular.

The electrodes may be arranged as interdigitated elongate fingers along the contact surface. In some examples the first electrode lies beneath the contact surface and is insulated from the surface by the dielectric of the head, for example by the insulator described above in relation to FIGS. 1 to 5. The first electrode may be insulated from the contact surface by a thickness of dielectric of at least 0.1 mm, for example at least 0.2 mm or 0.3 mm. In some possibilities the first electrode is insulated from the contact surface by a thickness of dielectric of less than 2 mm, for example less than 1.5 mm, for example less than 1 mm. The dielectric preferably comprises ceramic, and in some cases comprises shapal, and in some cases could also be the insulator described above in relation to FIGS. 1 to 5.

Although the electrodes may be straight, in some cases they may also be arranged in other shapes such as serpentine configurations or spirals along the contact surface. In some examples the electrodes are arranged along the contact surface to define the boundaries of concentric laminae. The laminae may be selected from the list comprising one of: circular; elliptical; square; polygonal rectangular; and irregular and the electrodes may define closed boundaries or they may define non-continuous open boundaries.

The above embodiments are to be understood as illustrative examples. Further embodiments are envisaged. It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

In some examples, one or more memory elements can store data and/or program instructions used to implement the operations described herein. Embodiments of the disclosure provide tangible, non-transitory storage media comprising program instructions operable to program a processor to perform any one or more of the methods described and/or claimed herein and/or to provide data processing apparatus as described and/or claimed herein.

1. A high dielectric strength insulator for use in insulating an electrode for a cold plasma generator, the high dielectric strength insulator comprising:
a base material having a high dielectric strength of at least 70 kV/mm; and
a coating layer formed on the base material, wherein the coating layer is at least one of:
(i) formed from a material having a dielectric strength equal to or greater than the base material;
(ii) formed from a material having a surface hardness greater than that of the base material; and
(iii) non-porous.

2. The high dielectric strength insulator of claim 1 wherein the coating layer is impermeable to water.

3. The high dielectric strength insulator of any of the previous claims wherein the coating layer has a surface hardness greater than 60 GPa, for example greater than or equal to 100 GPa.

4. The high dielectric strength insulator of any of the previous claims wherein the base material has a surface hardness less than 40 GPa, for example less than 1 GPa, for example less than or equal to 0.15 GPa.

5. The high dielectric strength insulator of any of the previous claims wherein the base material has a dielectric strength greater than 70 kV for example greater than or equal to 95 kV/mm.

6. The high dielectric strength insulator of any of the previous claims wherein the base material comprises boron nitride, optionally wherein the boron nitride base material is grade BO boron nitride, optionally wherein the boron nitride base material is parallel pressed.

7. The high dielectric strength insulator of any of the previous claims wherein the coating layer comprises natural diamond and/or synthetic diamond.

8. The high dielectric strength insulator of any of the previous claims wherein the coating layer comprises silicon dioxide, optionally wherein the silicon dioxide has a purity that is greater than 99.9999%, optionally wherein the silicon dioxide has a purity that is equal to or greater than 99.99999%.

9. The high dielectric strength insulator of any of the previous claims wherein the coating layer is formed as a thin film layer, for example at least 2 μm thick, optionally between 10 and 30 μm thick.

10. The high dielectric strength insulator of any of the previous claims wherein the high dielectric strength insulator further comprises a shield layer formed on the coating layer, the surface layer being at least one of (i) non-porous to oxygen, (ii) having a hardness greater than the base material and/or the coating layer, and (iii) having a dielectric strength equal to or greater than the base material and/or the coating layer.

11. The high dielectric strength insulator of claim 10 wherein the shield layer comprises silicon dioxide, optionally wherein the silicon dioxide in the shield layer has a purity that is greater than 99.9999%, optionally wherein the silicon dioxide in the shield layer has a purity that is equal to or greater than 99.99999%.

12. The high dielectric strength insulator of any of claim 10 or 11 wherein the shield layer is at least 2 μm thick, optionally between 10 and 30 μm thick.

13. The high dielectric strength insulator of any of the previous claims comprising at least one of (i) a plurality of surface ripples and (ii) a plurality of surface undulations shaped to lengthen the path of any linear tracking lines on the surface of the insulator.

14. A method of manufacturing a high dielectric strength insulator, the method comprising:
forming a base material from boron nitride;
coating the base material with a coating layer while the base material is held at an elevated temperature to inhibit moisture absorption, wherein the coating layer is at least one of:
(i) formed from a material having a dielectric strength equal to or greater than the base material; and
(ii) formed from a material having a surface hardness greater than that of the base material.
(iii) non-porous.

15. The method of claim 14 wherein the coating layer is impermeable to water.

16. The method of claim 14 or 15 wherein forming the base material further comprises at least one of:
(a) removing sharp corners and edges in the base material to inhibit the formation of high electromagnetic fields which cause high stress points in the material under high voltage conditions; and
(b) forming at least one of (i) a plurality of surface ripples and (ii) a plurality of surface undulations shaped to lengthen the path of any linear tracking lines on the surface of the base material.

17. The method of any of claims 14 to 16 further comprising cleaning the base material with a non-aqueous fluid to remove surface contamination after forming the base material, optionally wherein cleaning the base material further comprises cleaning the base material in an ultrasonic bath.

18. The method of any of claims 14 to 17 further comprising baking the base material to remove water from the base material prior to coating the base material with a coating layer, optionally wherein baking the base material comprises at least one of: (i) baking the base material at a temperature of at least 130° C. for at least 30 minutes, and (ii) baking the base material at a temperature of at least 150° C. for at least 30 minutes.

19. The method of any of claims 14 to 18 wherein the boron nitride base material is at least one of: (i) grade BO boron nitride, and (ii) parallel pressed.

20. The method of any of claims 14 to 19 wherein the coating layer comprises at least one of: (i) natural diamond, (ii) synthetic diamond and (iii) silicon dioxide, optionally wherein the coating layer is a thin film layer, for example at least 2 μm thick, optionally between 10 and 30 μm thick.

21. The method of any of claims 14 to 20 further comprising coating the coating layer with a shield layer formed on the coating layer, the surface layer being at least one of (i) non-porous, (ii) having a hardness greater than the base material and/or the coating layer, and (iii) having a dielectric strength equal to or greater than the base material and/or the coating layer.

22. The method of any of claims 14 to 21 wherein the shield layer is at least 2 μm thick, optionally between 10 and 30 μm thick.

23. A system for cold plasma generation, the system comprising:
(a) an electrode; and
(b) an insulator comprising:
a base material having a high dielectric strength of at least 70 kV/mm; and
a coating layer formed on the base material, wherein the coating layer is at least one of:
(i) formed from a material having a dielectric strength equal to or greater than the base material;
(ii) formed from a material having a surface hardness greater than that of the base material; and
(iii) non-porous.

24. The system of claim 23 wherein the electrode comprises a first set of electrodes and a second set of electrodes each comprising a plurality of electrodes, and wherein each electrode of a set is arranged in the same plane as the other electrodes of its set.

25. The system of claim 24 wherein the electrodes of the first set of electrodes are interdigitated with the electrodes of the second set of electrodes.
